(19) United States
(12) Patent Application Publication
(10) Pub. No.: US 2022/0033373 A1
(43) Pub. Date: Feb. 3, 2022

(54) METHODS FOR SELECTIVE AROMATIZATION OF CANNABINOIDS

(71) Applicant: Kentucky Blu LLC, Franklin, KY (US)

(72) Inventors: Maxwell B. Loewinger, Bowling Green, KY (US); David H. Young, Bowling Green, KY (US); Ryan T. St. Clair, Louisville, KY (US)

(73) Assignee: Kentucky Blu LLC, Franklin, KY (US)

(21) Appl. No.: 17/390,874

(22) Filed: Jul. 31, 2021

Related U.S. Application Data
(60) Provisional application No. 63/060,183, filed on Aug. 3, 2020.

(51) Int. Cl. C07D 311/80

(52) U.S. Cl. CPC C07D 311/80

(57) ABSTRACT

The present invention relates to methods of selective aromatization of cannabinoids. Such methods may be used, among other purposes, for the removal of delta-9-tetrahydrocannabinol from hemp extracts or other samples by selectively converting delta-9-tetrahydrocannabinol to cannabinol using ortho-quinone catalysts.

### CROSS-REFERENCE TO RELATED APPLICATION

This application claims the benefit of priority to U.S. provisional patent application Ser. No. 63/060,183, filed Aug. 3, 2020, for METHODS FOR SELECTIVE AROMATIZATION OF CANNABINOIDS, incorporated herein by reference.

### FIELD

The present invention relates to methods of selective aromatization of cannabinoids. Such methods may be used, among other purposes, for the removal of delta-9-tetrahydrocannabinol from hemp extracts or other samples by selectively converting delta-9-tetrahydrocannabinol to cannabinol using ortho-quinone catalysts.

### BACKGROUND

Industrial hemp has served as a source of fiber and oilseed worldwide for centuries, producing industrial and consumer products. In the 1600's, hemp came to North America and played an important role in agriculture. The early days of hemp included low tetrahydrocannabinol (THC) content plants that were traditionally grown as a fiber source. However, as the industry evolved so did the higher THC content “marijuana” hemp varieties from southern Asia. Industrial hemp and marijuana, botanically, are from the same species of plant, Cannabis sativa. Due to the use of marijuana plants in the drug trade, industrial hemp was linked with its higher THC cousin in the 1930s with legislation at both the state and federal level that banned their cultivation.

After passage of the Agriculture Improvement Act of 2018 (also referred to as the 2018 Farm Bill), hemp and marijuana are treated differently under federal law. The 2018 Farm Bill defines hemp as the plant Cannabis sativa and any part thereof with a delta-9-tetrahydrocannabinol (D9-THC) concentration of not more than 0.3% by dry weight. THC is one of over 60 naturally occurring cannabinoids found in Cannabis sativa, but is the primary compound responsible for the psychoactive properties of the plant. A selection of naturally occurring cannabinoids is shown in FIG. 1. Other cannabinoids, including but not limited to cannabidiol (CBD), cannabinol (CBN), cannabidiolic acid (CBDA), cannabichromene (CBC), delta-8-tetrahydrocannabinol (D8-THC) and cannabigerol (CBG), are considered to have desirable properties. As such, cultivators of hemp are challenged to produce Cannabis sativa plants with D9-THC concentrations of not more than 0.3% by dry weight to prevent the plants from being classified as marijuana while maintaining or increasing concentrations of other desired cannabinoids. Even when hemp biomass meets this legal definition, subsequent extraction and refinement frequently increases the concentration of D9-THC above the 0.3% limit. This prevents the sanctioned sale of these concentrated extracts, presenting difficulties for hemp producers and processors. Currently, very few processes exist for the removal or remediation of D9-THC from hemp extracts, with chromatography being the current industry standard. Chromatographic separation has significant drawbacks in that it requires the use and distillation of large amounts of solvent, produces significant waste, is inherently low throughput, labor-intensive, and is costly to implement and perform.

A potential alternative remediation method is the degradation of D9-THC to CBN or other non-psychoactive cannabinoid(s). Methods for converting D9-THC to CBN using elemental sulfur, selenium, palladium (on carbon), platinum, iodine, or p-chloranil, have been reported. However, these methods all suffer from serious drawbacks, including harsh conditions, noxious byproducts, high expense and low yields. Most notably, none are well suited to the remediation of D9-THC from hemp extracts containing complex mixtures of cannabinoids, due to these reagents' cross-reactivity with CBD, CBC, D8-THC, CBG, and others.

The inventors of the present disclosure realized that methods are needed to remove or remediate D9-THC in a simple, specific, scalable, and cost-effective manner. Certain preferred features of the present disclosure address these and other needs and provide other important advantages.

### SUMMARY

The instant invention concerns novel methods for the oxidation of D9-THC to CBN using ortho-quinones, as well as structurally related compounds such as enzyme cofactors and ortho-iminoquinones, as stoichiometric or catalytic oxidants. These methods permit efficient production of CBN from D9-THC and a simple procedure for the decomposition of D9-THC from hemp extracts.

The chemistry of quinone oxidants has historically been focused on para-quinones (abbreviated as p-quinones), which are typically more stable and easier to prepare than ortho-quinones (abbreviated as o-quinones). However, o-quinones with a broad range of substituents and oxidation potentials have been found by the inventors to convert D9-THC to CBN under mild conditions. In contrast, only high-potential p-quinones bearing electron withdrawing groups oxidize D9-THC to CBN, and only under forcing conditions. This provides several advantages to o-quinone chemistry over p-quinone chemistry, including (1) selectivity, as low-potential o-quinones can selectively degrade different cannabinoids while high-potential p-quinones are not selective between cannabinoids; (2) simplicity, as the cannabinoid aromatization reaction using o-quinones can be performed at or near room temperature while use of p-quinones requires significantly elevated temperatures deleterious to yields and product distributions; (3) safety, as the higher temperatures and more powerful oxidants required for p-quinones present occupational and environmental hazards, and quinones with higher redox potentials tend to be more toxic; (4) clarity, as a byproduct of the D9-THC to CBN reaction using p-chloranil catalysts is the corresponding hydroquinone, and hydroquinone is a known environmental transformation product of the herbicide aclonifen, such that use of the p-chloranil p-quinone catalyst in a D9-THC to CBN reaction could inaccurately indicate that the originating Cannabis plant was treated with the herbicide; (5) tunability, as multiple o-quinones efficiently dehydrogenate THC, so that particular o-quinones can be chosen for their selectivity, reactivity, product distribution, solubility characteristics, etc.; and (6) catalysis, as high potential quinones are not amendable to catalytic turnover with a sacrificial oxidant because this would require an even stronger oxidant. In contrast, a low potential quinone catalyst may be reoxidized in situ with an oxidant mild enough not to undergo significant side-reactions with the substrates.

At its simplest, aromatization challenges the substrate with an oxidant of some strength and the energy required to achieve the reaction barrier. By basic thermodynamics, stronger oxidants would be expected to be more reactive than weaker ones, under the same conditions. The present invention unexpectedly discloses different reaction outcomes from oxidants of similar strength (e.g., o-chloranil vs. p-chloranil) and unexpectedly discloses low-potential oxidants achieving oxidation under conditions where high potential oxidants are unreactive. The disclosed methods employ low-reduction potential oxidants to offer a simple, highly efficient conversion of D9-THC to CBN under mild conditions (e.g., room temperature), at high concentrations, in a scalable, inexpensive transformation. The reaction proceeds with high selectivity, as D9-THC may be decomposed to CBN even in the presence of other desirable cannabinoids such as CBD, CBDA, CBC, D8-THC, and CBG, without observable decomposition of these other cannabinoids.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein does not necessarily address every object described herein, and each embodiment does not necessarily include each feature described. Other forms, embodiments, objects, advantages, benefits, features, and aspects of the present disclosure will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

Some of the figures shown herein may include dimensions or may have been created from scaled drawings. However, such dimensions, or the relative scaling within a figure, are by way of example only, and are not to be construed as limiting the scope of this invention.

FIG. 1 depicts a selection of naturally occurring cannabinoids found in hemp. While the R-group is shown as a five carbon aliphatic chain, it should be understood that other side chains, aliphatic or otherwise, are also within the scope of this invention.

FIG. 2 depicts the chemical equation for production of CBN from CBD or D8-THC.

FIG. 3 depicts the chemical equation for conversion of D9-THC to CBN using a low potential quinone catalyst.

FIG. 4 depicts the chemical equation for conversion of D9-THC to CBN using a high potential quinone catalyst.

FIG. 5 depicts the chemical equation for oxidation of D9-THC in a mixture containing CBD.

FIG. 6 depicts the chemical equation for catalytic oxidation of D9-THC.

FIG. 7 depicts the chemical equation for conversion of CBD to CBN using a low potential quinone catalyst.

FIG. 8 depicts the chemical equation for conversion of CBD to CBN using a low potential quinone catalyst and a sacrificial oxidant.

FIG. 9 depicts the chemical equation for conversion of D8-THC to CBN.

FIG. 10 depicts the chemical equation for conversion of delta-9-tetrahydrocannabivarin to cannabivarin and dihydrocannabivarin.

FIG. 11 depicts the chemical equation for conversion of D9-THC to dihydrocannabinol.

FIG. 12 depicts natural cannabinoids with varied chain lengths.

FIG. 13 depicts the chemical equation for production of AM1710 from its tetrahydrogenated analog.

FIG. 14 depicts production of cannabinol monomethyl ether from D9-THC monomethyl ether.

FIG. 15 depicts production of 11-hydroxy-cannabinol from 11-hydroxy-D9-THC.

FIG. 16 depicts chemical equations for production of HU-345.

FIG. 17 depicts families of catalysts and redox tautomers.

FIG. 18 depicts exemplary quinones.

FIG. 19 depicts redox regeneration of the catalytic oxidant ortho-quinone from its reduced catechol using p-chloranil.

### DETAILED DESCRIPTION OF THE ILLUSTRATED EMBODIMENTS

For the purposes of promoting an understanding of the principles of the invention disclosed herein, reference will now be made to one or more embodiments, which may or may not be illustrated in the drawings, and specific language will be used to describe the same. It will nevertheless be understood that no limitation of the scope of the disclosure is thereby intended; any alterations and further modifications of the described or illustrated embodiments, and any further applications of the principles of the disclosure as illustrated herein are contemplated as would normally occur to one skilled in the art to which the disclosure relates. At least one embodiment of the disclosure is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to benefits or advantages provided by some embodiments, other embodiments may not include those same benefits or advantages, or may include different benefits or advantages. Any benefits or advantages described herein are not to be construed as limiting to any of the claims.

Specific quantities (spatial dimensions, temperatures, pressures, times, force, resistance, current, voltage, concentrations, wavelengths, frequencies, heat transfer coefficients, dimensionless parameters, volumes, etc.) may be used explicitly or implicitly herein; such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated.

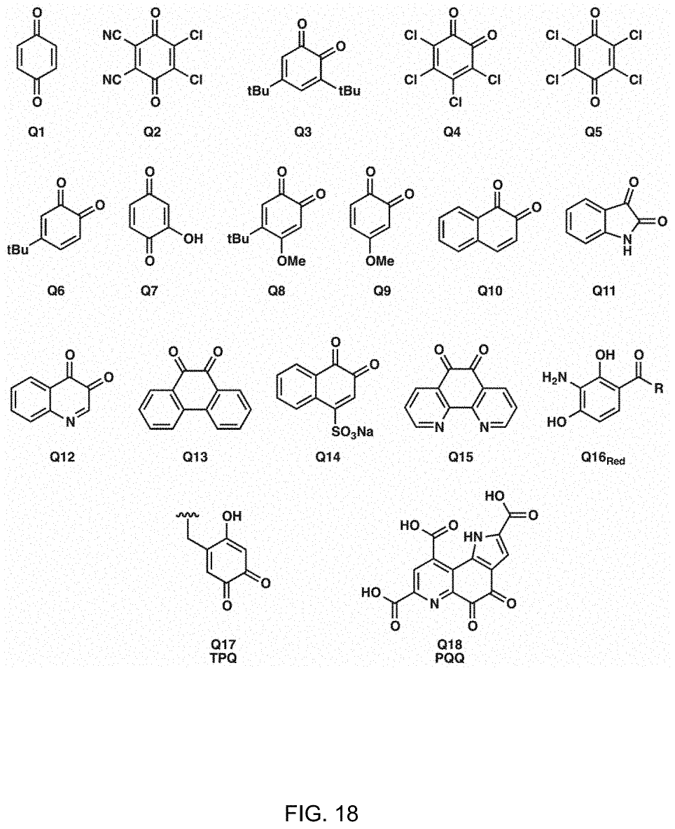

Various quinones are abbreviated herein according to the following table and as shown in FIG. 18.

TABLE 1
Quinone abbreviations

| Quinone | Abbreviation |
|---|---|
| benzoquinone | Q1 |
| 2,3-dichloro-5,6-benzoquinone | Q2 |
| 3,5-di-tert-butyl-1,2-benzoquinone | Q3 |
| ortho-chloranil | Q4 |
| p-chloranil | Q5 |
| 5-tert-butyl-1,2-benzoquinone | Q6 |
| 3-hydroxy-benzoquinone | Q7 |
| 4-methoxy-5-tert-butyl-1,2-benzoquinone | Q8 |
| 4-methoxy-1,2-benzoquinone | Q9 |
| 1,2-naphthaquinone | Q10 |
| isatin | Q11 |
| Quinoline-3,4-dione | Q12 |
| Phenanthrene-9,10-dione | Q13 |
| 1,2,-naphthaquinone-4-sulfonic acid sodium salt | Q14 |
| 1,10-phenanthroline-5,6-dione | Q15 |
| 3-carbonyl-2,6-dihydroxyaniline | Q16 |
| topaquinone | Q17, TPQ |
| pyrroloquinoline quinone | Q18, PQQ |

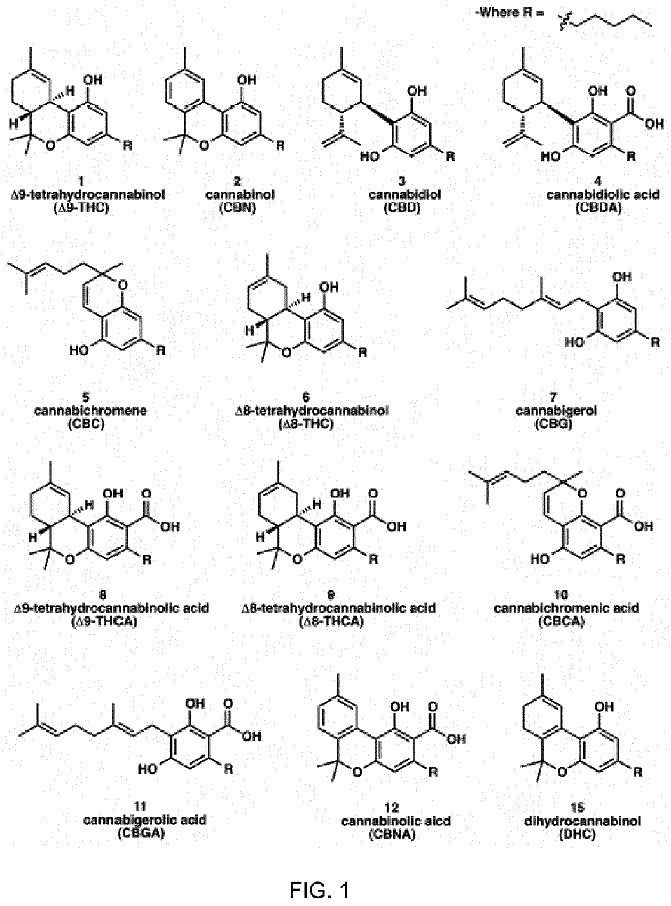

Embodiments of the present invention include a selective process for conversion of D9-THC to CBN using ortho-quinones, as well as structurally related compounds such as enzyme cofactors and ortho-iminoquinones, as stoichiometric or catalytic oxidants, as shown in FIG. 1. This method may be applied to pure D9-THC for the production of CBN, or in situ to hemp extracts for the selective conversion of D9-THC to CBN in the presence of other cannabinoids, without substantial degradation of those cannabinoids. The family of ortho-quinone (and related) oxidants can be applied stoichiometrically (200 mol % or greater) with respect to D9-THC or catalytically (200 mol % to 10 mol % or lower) through inclusion of a sacrificial oxidant such as p-chloranil (Q5) and/or O₂ and others. Quinones referred to as “high potential” refer to those with oxidation potentials generally greater than benzoquinone, while quinones referred to as “low potential” refer to those with oxidation potentials generally lower than benzoquinone.

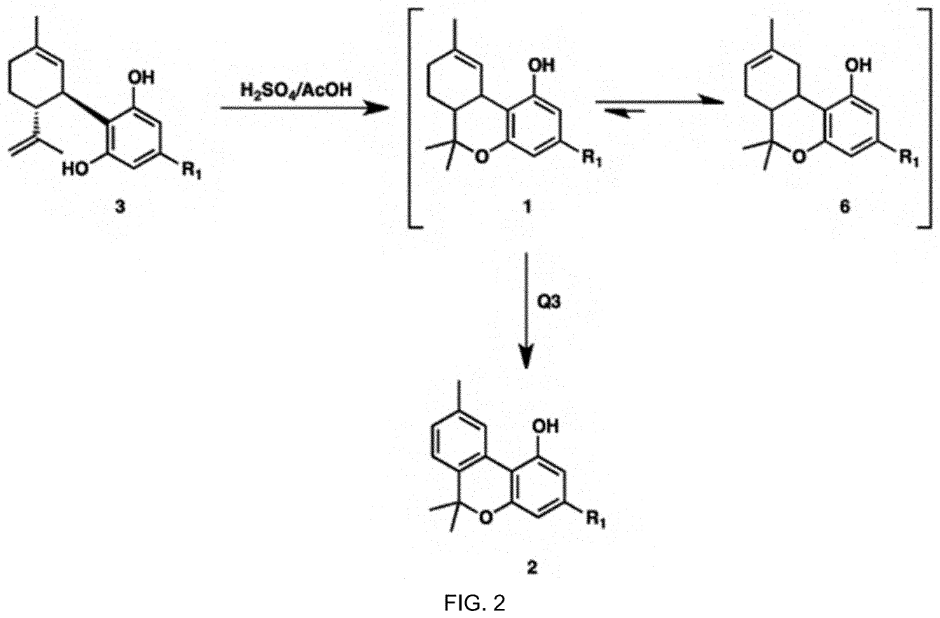

Further, these catalysts may be used to convert either CBD or D8-THC to CBN through a one-pot tandem cyclization/isomerization system that does not lead to the observable generation of significant quantities of D9-THC using the equation shown in FIG. 2. This transformation may be performed either stoichiometrically or catalytically.

General Method for Monitoring Reaction Via HPLC Analysis

Aliquots are taken for in process control in the following manner: ˜100 μL of the reaction mixture is charged to a tared vial and the mass is recorded. To the vial is added 5 mL of ethyl acetate (EtOAc) and the mixture is vortexed to achieve dissolution. 3 mL of saturated sodium bisulfite are added and the mixture is vortexed until a complete color change is observed. After separation of the resulting biphasic mixture, the organic phase is decanted and washed with saturated sodium bicarbonate. After the resulting biphasic mixture has separated, 1 mL of the organic fraction is decanted for HPLC analysis.

General Procedures

General Method for Purification of Products

After HPLC indicates reaction completion, the reaction mixture is diluted in 10 volumes ethyl acetate and washed with sodium bisulfite, then sodium bicarbonate, then water, then brine. The organic layer is dried over a drying agent such as, for example, sodium sulfate or magnesium sulfate, then concentrated under reduced pressure. The residue is purified via distillation or through a plug of silica gel.

Method for Reduction of Ortho-Quinones with Sodium Bisulfite

To a flask is added 1.00 g of 3,5-di-tert-butyl-1,2-benzoquinone (Q3) (4.54 mmol, 1 equivalent) with 10 ml of heptane. This suspension is briefly stirred, then 18.7 ml saturated sodium bisulfite (75.33 mmol, 16.6 equivalents) are added and the biphasic mixture is stirred vigorously overnight. After 24 hours, the reaction contents are extracted with 25 mL EtOAc and the organic phase is washed with saturated sodium bicarbonate, then water, then brine. The organic phase is dried over sodium sulfate then concentrated in vacuo to yield 0.51 g catechol Q3_{Red} as a light purple solid in 50% yield.

Oxidation of D9-THC to CBN with a Low Potential Ortho-Quinone Catalyst Q3

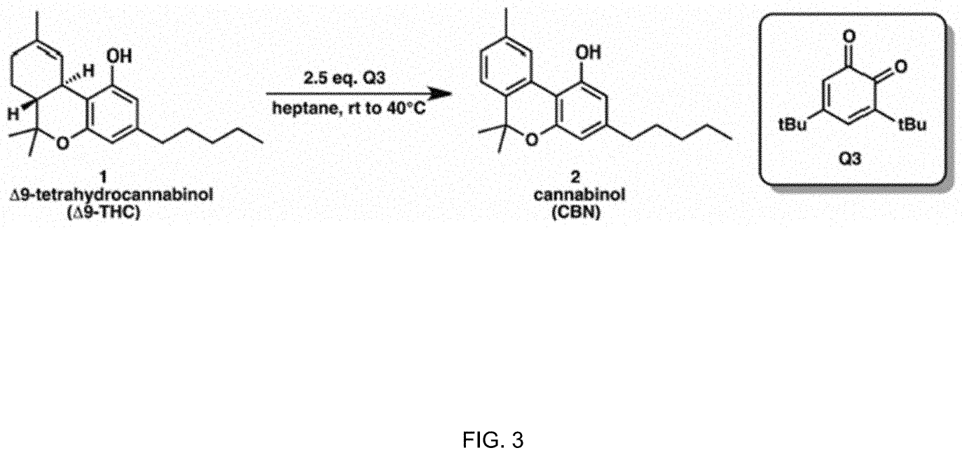

Referring now to FIG. 3, to a stirred solution of D9-THC (100 mg, 0.318 mmol, 1 equivalent) in 1 mL of heptane is added 3,5-di-tert-butyl-1,2-benzoquinone (Q3) (175 mg, 0.795 mmol, 2.5 equivalents) and the reaction is stirred at room temperature. As used herein, the term room temperature (“rt” in the figures) refers to a range of temperatures from 20° C. to 27° C. In other embodiments, the reaction occurs at a temperature within the range of room temperature to 40° C. (i.e., between 20° C. and 40° C.). In certain embodiments, the reaction occurs at 22° C.

After 24 hours, HPLC indicates reaction completion. The reaction is purified as described in the general procedure. The residue is distilled to afford CBN.

Oxidation of D9-THC to CBN with a High Potential Ortho-Quinone Catalyst Q4

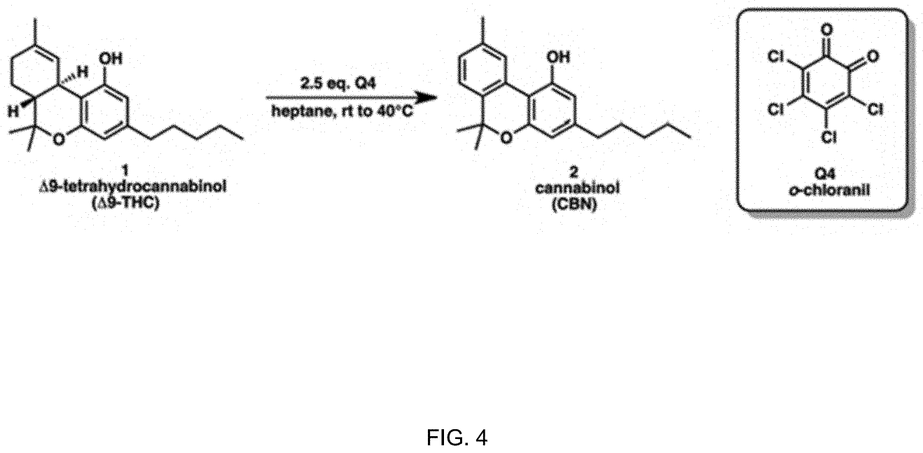

Referring now to FIG. 4, to a stirred solution of THC (100 mg, 0.318 mmol, 1 equivalent) in 1 mL of heptane is added ortho-chloranil (Q4) (195 mg, 0.795 mmol, 2.5 equivalents) and the reaction is stirred at room temperature. Aliquots for HPLC analysis are taken as described above in the general procedures.

After 4 hours, HPLC analysis indicates the reaction is complete. The reaction is worked up as described in the general procedures, yielding CBN as a red oil after distillation.

Remediation of D9-THC in the Presence of CBD with a Low Potential Quinone

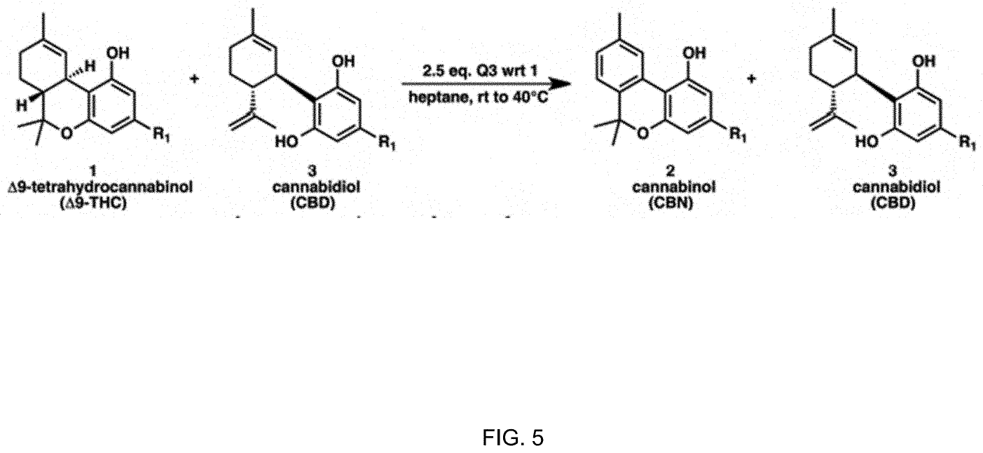

Referring now to FIG. 5, to a flask containing CBD (494 mg, 1.570 mmol, 19 equivalents) is added D9-THC (26 mg, 0.083 mmol, 1 equivalent) dissolved in 0.75 ml heptane, and the reaction is stirred to achieve dissolution. Subsequently, 46 mg 3,5-di-tert-butyl-1,2-benzoquinone (Q3) (46 mg, 0.2075 mmol, 2.5 equivalents wrt to THC) is added. The reaction is stirred overnight. Aliquots are taken as described in the general procedure for in process monitoring by HPLC.

After 19 hours, HPLC analysis indicates complete consumption of D9-THC. After 24 hours, HPLC indicates reaction completion. The reaction is purified as described in the general procedure. The residue is distilled to afford a yellow oil containing CBN and CBD. HPLC chromatograms indicating the presence of D9-THC in the produced solution are not observable.

Remediation of D9-THC in the Presence of CBD in Crude, Decarboxylated Extract with a Low Potential Quinone

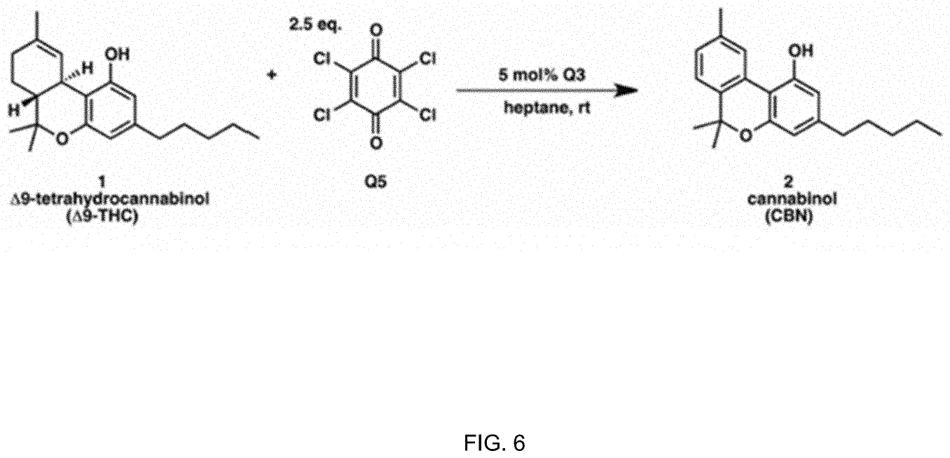

Referring now to FIG. 6, to a flask containing 5.15 g decarboxylated, winterized (i.e., soaked in alcohol, frozen to separate out lipids, then returned to room temperature) crude hemp extract (2.75% D9-THC [0.36 mmol, 1 equivalent], 0.02% CBN, 60.3% CBD, 2.76% CBC) is added 7.725 ml heptane (1.5 volumes with respect to total extract) followed by 205 mg 3,5-di-tert-butyl-1,2-benzoquinone (Q3) (0.90 mmol, 2.5 equivalents with respect to 1). The reaction is heated to 40° C. and stirred. Aliquots are taken as described in the general procedure for in-process monitoring by HPLC.

After 24 hours, HPLC analysis indicates reaction is complete. The products are purified as described in the general procedure. The resulting residue is distilled to produce a yellow oil containing CBN, CBD, and CBC, with peaks indicating presence of D9-THC not observable by HPLC analysis.

Synthesis of CBN from D9-THC Using Catalytic Ortho-Quinone in the Presence of a Sacrificial Oxidant

Referring now to FIG. 6, to a flask containing 1.000 g D9-THC (3.18 mmol, 1 equivalent) diluted in 10 mL methylene chloride is added 1.720 g Q5 (7.00 mmol, 2.2 equivalents) followed by 35 mg 3,5-di-tert-butyl-1,2-benzoquinone (Q3) (0.159 mmol, 5 mol %). The reaction is stirred at room temperature. Aliquots are taken as described in the general procedure for in-process monitoring by HPLC.

After 24 hours, HPLC analysis indicates the reaction is complete by complete disappearance of peak corresponding to D9-THC. The products are purified as described in the general procedure. The resulting residue is distilled to yield CBN as a red oil.

Synthesis of CBN from CBD Through One-Pot, Tandem Cyclization/Oxidation Using a Low Potential Ortho-Quinone

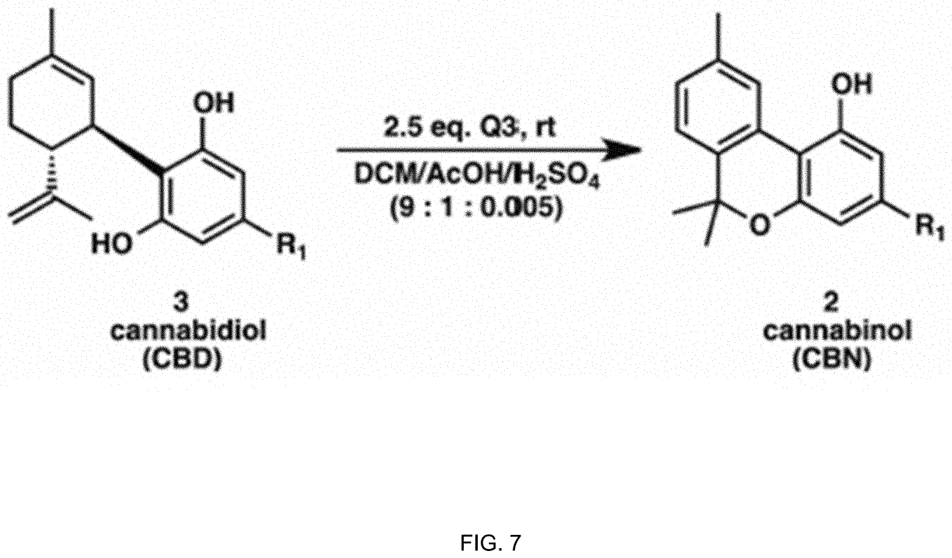

Referring now to FIG. 7, to a flask is added 100 mg CBD (0.318 mmol, 1 equivalent), dissolved in 1 ml dichloromethane (DCM). The flask is charged with 175 mg 3,5-di-tert-butyl-1,2-benzoquinone (Q3) (0.795 mmol, 2.5 equivalents) and the reaction is stirred. To the stirring flask is added 100 μL of 0.1M sulfuric acid (H₂SO₄) in glacial (i.e., anhydrous) acetic acid (AcOH). Reaction stirred. Aliquots are taken as described in the general procedure for in-process monitoring by HPLC.

After 24 hours, HPLC analysis indicates the reaction is complete by complete disappearance of peak corresponding to CBD. The products are purified as described in the general procedure and the residue distilled to yield CBN as a red oil. Throughout the transformation, concentrations of D9-THC remain below 0.3%.

Reviewing this reaction as compared to the reactions shown in FIG. 2 and FIG. 5, AcOH and H₂SO₄ convert CBD to D9-THC, then Q3 catalyzes the conversion of D9-THC to CBN. By omitting AcOH and H₂SO₄ in the reaction shown in FIG. 5, D9-THC may be converted to CBN via Q3 in the presence of CBD without also converting CBD to CBN. The Q3 mediated reaction may be performed in DCM, heptane, or other appropriate solvent. However, experimental results have shown that the two-step, single flask, reaction shown in FIG. 7 utilizing AcOH and H₂SO₄ is preferably performed in DCM as performing the aromatization in heptane results in the formation of an undesired side-product.

Catalytic Synthesis of CBN from CBD Through One-Pot, Tandem Cyclization/Oxidation Using a Low Potential Ortho-Quinone in the Presence of a Sacrificial Oxidant Q5

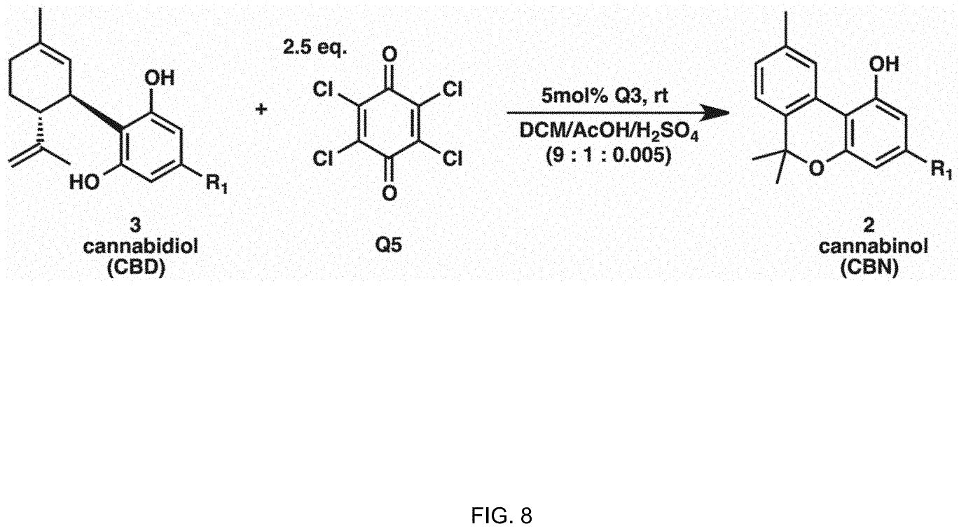

Referring now to FIG. 8, the described conversion of CBD to CBN in the presence of stoichiometric 3,5-di-tert-butyl-1,2-benzoquinone (Q3) is performed catalytically through a modified procedure:

To a flask is added 1.00 g CBD (3.18 mmol, 1 equivalent), dissolved in 10 ml DCM. The flask is charged with 1.95 g p-chloroanil (Q5) (7.95 mmol, 2.5 equivalents) followed by 35 mg 3,5-di-tert-butyl-1,2-benzoquinone (Q3) (0.16 mmol, 5 mol %) and the reaction is stirred. To the stirring flask is added 1.0 mL of 0.1M H₂SO₄ in glacial acetic acid. Reaction is stirred. Aliquots are taken for HPLC analysis as described in the general procedure.

After 24 hours, HPLC analysis indicates the reaction is complete by complete disappearance of peak corresponding to CBD. The products are purified as described in the general procedure and the produced residue is to yield CBN as a red oil. Throughout the transformation, concentrations of D9-THC remain below 0.3%.

Synthesis of CBN from D8-THC Through One-Pot, Tandem Isomerization/Oxidation Using a Low Potential Ortho-Quinone

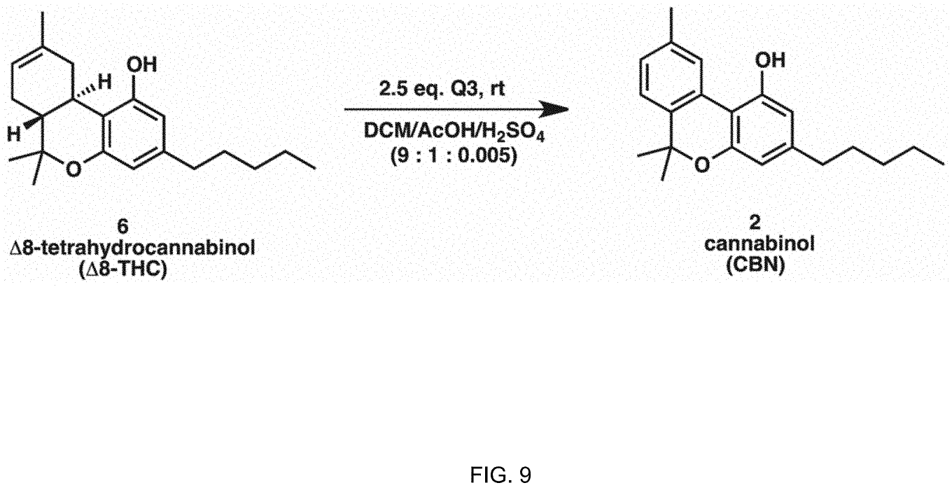

Referring now to FIG. 9, to a flask is added 100 mg D8-THC (0.318 mmol, 1 equivalent), dissolved in 1 ml DCM. The flask is charged with 175 mg 3,5-di-tert-butyl-1,2-benzoquinone (Q3) (0.795 mmol, 2.5 equivalents) and the reaction is stirred. To the stirring flask is added 100 μL of 0.1M H₂SO₄ in glacial (i.e., anhydrous) acetic acid. Reaction mixture stirred. Aliquots are taken for HPLC analysis as described in the general procedure.

After 24 hours, HPLC analysis indicates the reaction is complete by complete disappearance of peak corresponding to D8-THC. The products are purified as described in the general procedure and the produced residue is to yield CBN as a red oil. Throughout the transformation, concentrations of D9-THC remain below 0.3%.

Synthesis of Cannabivarin from Tetrahydrocannabivarin

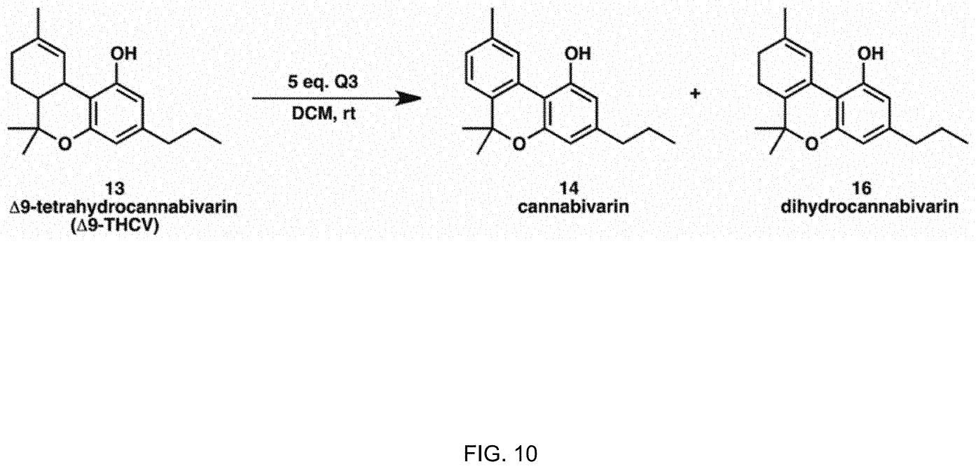

Referring now to FIG. 10, to a vial is charged 1 mL of tetrahydrocannabivarin (THCV) analytical standard solution (0.05 mg/ml in MeOH). Solvent is removed under a steady stream of dry air. Once concentrated, 200 μL DCM containing 0.2 mg 3,5-di-tert-butyl-1,2-benzoquinone (Q3) (7.5 μmol, 5 eq.) is added to the vial and the vial is stirred overnight at room temperature.

After 18 h, the flask is treated with a few drops of sat. NaHSO₃, and stirred. Then the solvent is removed under a steady stream of dry air. The residue is extracted with 0.5 ml EtOAc and the product submitted to HPLC analysis. HPLC analysis indicates the complete disappearance of the peak corresponding to THCV (2.389 min.) and the emergence of the peak corresponding to cannabivarin (CBV) (2.188 min.) as well as a peak characteristic of dihydrocannabinol derivatives, assigned to diene dihydrocannabivarin (2.650 min.).

Preparation of Dihydrocannabinol from D9-THC

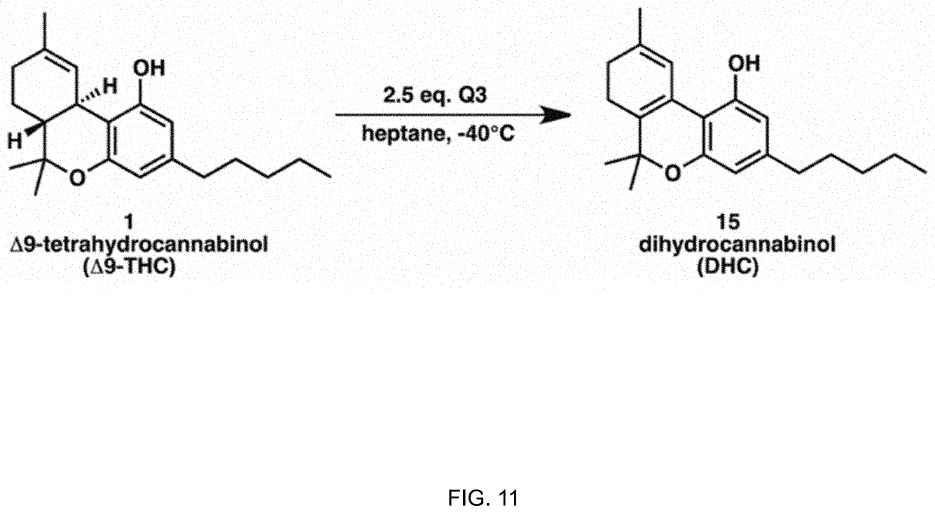

Referring now to FIG. 11, to a stirred vial containing 100 mg of D9-THC (0.318 mmol, 1 equivalent) in 1 mL of heptane, cooled to −40° C., is added 210 mg 3,5-di-tert-butyl-1,2-benzoquinone (Q3) (0.954 mmol, 3 equivalents). In some embodiments, the Q3 is added all at once, rather than stepwise, to avoid introduction of water to the reaction. The reaction is stirred overnight while maintaining the temperature at −40° C. Aliquots are taken as described in the general procedure for in-process monitoring by HPLC.

After 18 h, HPLC analysis indicates the complete disappearance of D9-THC and replacement with a closely off-set eluting peak (4.260 min.) with strong absorbance at 304 nm. To the reaction mixture is added 2 mL of sodium bisulfite, and the flask is stirred at 0° C. for 1 h. After 1 h, the organic layer is decanted, washed with saturated NaHCO₃ (aq.), then H₂O, then saturated brine. The organic phase is dried over MgSO₄ (s), and concentrated in vacuo to yield crude dihydrocannabinol (DHC) as an orange oil.

Without being bound by theory, the oxidation of THC to CBN requires two sequential oxidations, whereby formation of the intermediate DHC is rapidly followed by the more facile oxidation to CBN such that DHC is not observed in cannabis extracts. By conducting this reaction at a very low temperature, the second oxidation was slowed, allowing the reaction to be quenched prior to complete aromatization and isolating the intermediate.

Discussion

Solvents

The conversion of D9-THC to CBN by 3,5-di-tert-butyl-1,2-benzoquinone (Q3) and related catalysts exhibits broad solvent scope. Specifically, experimental results have shown the reaction to proceed smoothly in acetonitrile, methylene chloride, toluene, methanol and ethanol, tetrahydrofuran, acetic acid, dimethyl sulfoxide (DMSO), chloroform, acetone, and aliphatic hydrocarbons such as heptane, producing CBN in high conversion.

Cannabinoids

The ortho-quinone catalytic system, with 3,5-di-tert-butyl-1,2-benzoquinone (Q3) as the representative oxidant, is highly selective for the dehydrogenation of the neutral tetrahydrocannabinols and their olefin positional isomers. However, the catalytic system can be smoothly applied to both naturally occurring and synthetic derivatives of tetrahydrocannabinol and their olefin positional isomers.

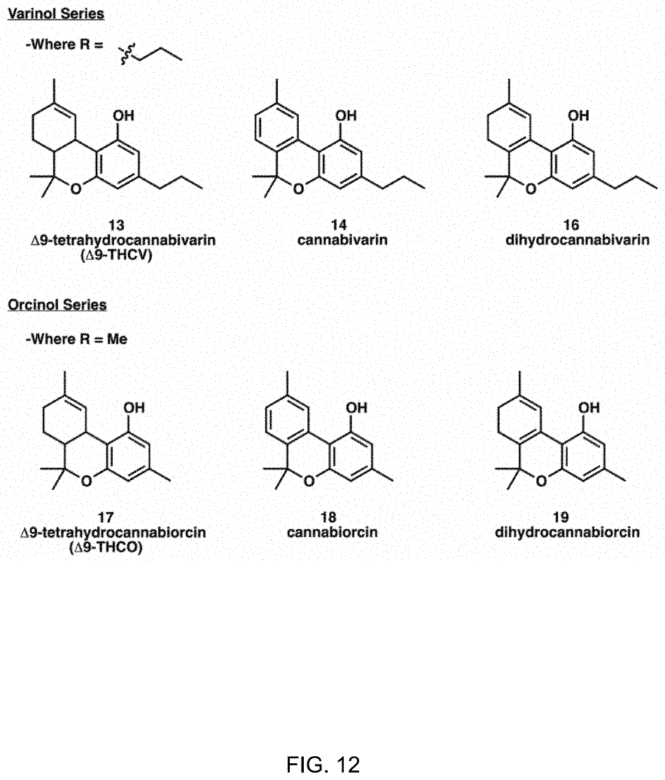

Natural variants of D9-THC have been isolated in nature with varying chain length. Several examples of such natural variants are shown in FIG. 12. In particular, linear alkyl substituents of odd numbered values (n=1, 3, 5, 7, 9, etc.) have been identified. As exemplified by the oxidation of THCV (n=3) to cannabivarin, natural cannabinoid analogs of D9-THC may be converted to their aromatic forms in the presence of 3,5-di-tert-butyl-1,2-benzoquinone (Q3).

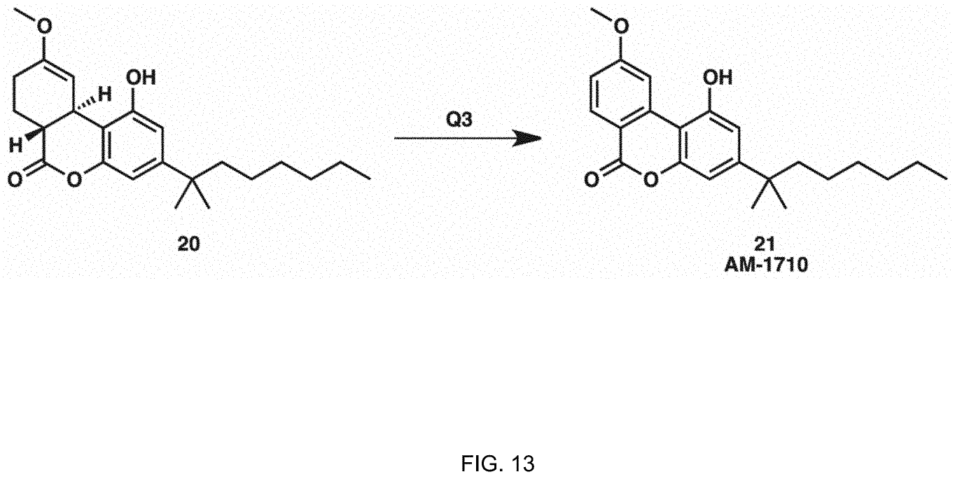

Synthetic analogs of D9-THC with other linear chain lengths and/or branched, cyclic, and aromatic side chains have been prepared. Dehydrogenation of these analogs proceeds smoothly to their aromatic products. For example, as shown in FIG. 13, the molecule AM1710, a cannabilactone cannabinoid receptor 2 agonist, may be synthesized from its tetrahydrogenated analog through dehydrogenation with 3,5-di-tert-butyl-1,2-benzoquinone (Q3).

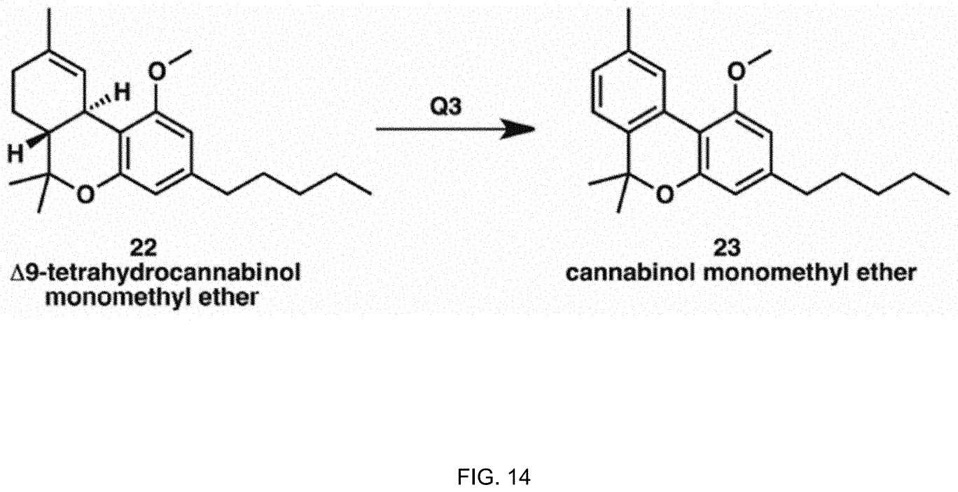

The reaction is tolerant to substitution and variation at the parent aromatic ring. As shown in FIG. 14, cannabinol monomethyl ether may be produced via Q3-mediated dehydrogenation from D9-THC monomethyl ether.

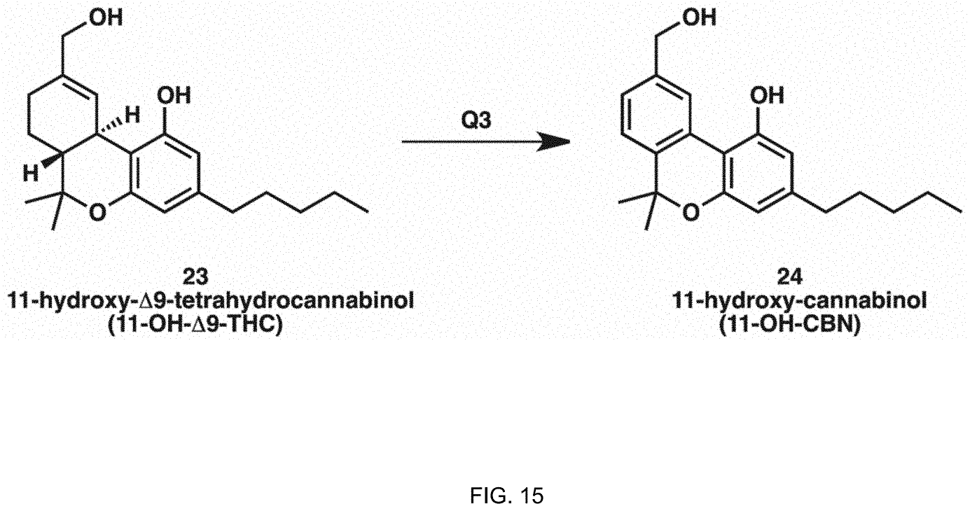

Biological oxidation products of THC are also oxidized providing facile access to metabolites of CBN. As shown in FIG. 15, treatment of a metabolite of THC, 11-hydroxy-delta-9-tetrahydrocannabinol (11-OH-D9-THC) with 3,5-di-tert-butyl-1,2-benzoquinone (Q3), yields the aromatized 11-hydroxy-cannabinol (11-OH—CBN).

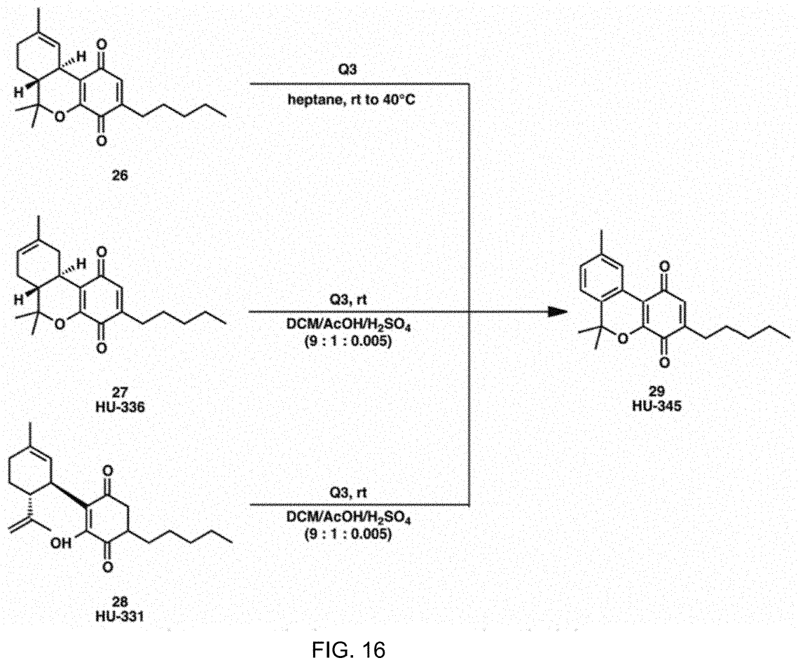

The one-pot, acid-mediated cyclization/oxidation and isomerization/oxidation cascades used to produce CBN from CBD and D8-THC, as respectively shown in FIGS. 7 and 9, can be used to produce aromatized cannabinoids from CBD- and D8-derivatives. For example, as shown in FIG. 16 the aortic ring angiogenesis inhibitor HU-3459 (cannabinol quinone) (29) may be produced from the tetrahydro derivative (26) under the previously described conditions. HU-345 (29) may also be produced from HU-336 (27) or HU-331 (28), via acid-mediated oxidation cascades.

Catalyst Selection

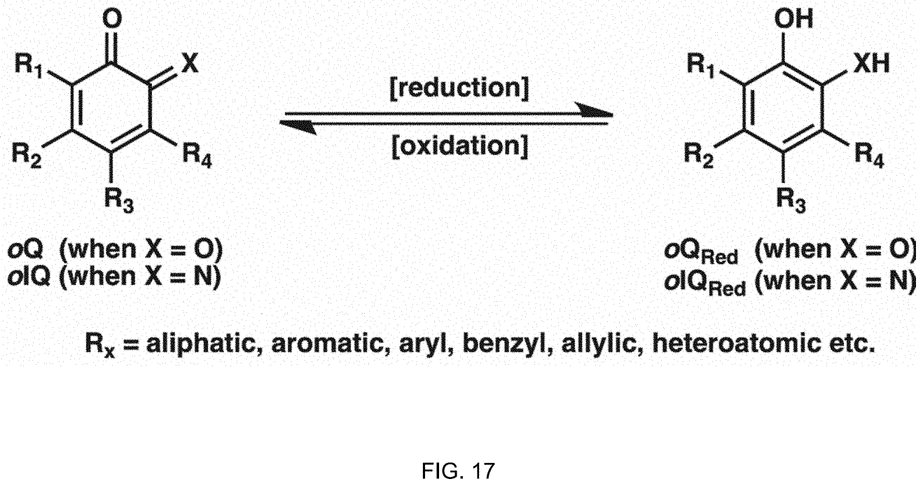

The reaction exhibits broad scope in catalyst selection from compounds possessing the ortho-quinone (oQ) structural motif. Referring now to FIG. 17, the reaction tolerates substituents at all positions of the aromatic ring (R1, R2, R3, R4), including electron-donating substituents, electron-withdrawing substituents, sterically bulky alkyl substituents, aromatic substituents and other functional groups. FIG. 18 displays exemplary quinones, wherein Q3, Q4, and Q6-Q18 are examples of ortho-quinones viable for use in the transformative methods disclosed herein. The ortho-iminoquinone family (oIQ) of catalysts represented generally in FIG. 17 may also be used in the disclosed methods.

Ortho-quinones and ortho-iminoquinones represent cofactors for a number of enzymes that facilitate biologically significant oxidations. Unbound and bound cofactors possessing either the oQ or oIQ structural motif, such as topaquinone (Q17, TPQ) and pyrroloquinoline quinone (Q18, PQQ), as well as enzymatic systems employing cofactors from these structural families, are also viable catalysts for dehydrogenation of D9-THC and analogous cannabinoids.

The active oQ or oIQ oxidant may be regenerated in situ using a sacrificial oxidant or secondary redox cycle (aerobic, enzymatic, photochemical, electrochemical, and/or biological). For this reason, the reduced forms of oQ-catalysts (catechols, oQ_{Red}) and oIQ (1,2-aminophenols, oIQ_{Red}) may also be selected as competent catalysts for the dehydrogenation of cannabinoids, especially in cases where redox coupling occurs.

Redox Coupling

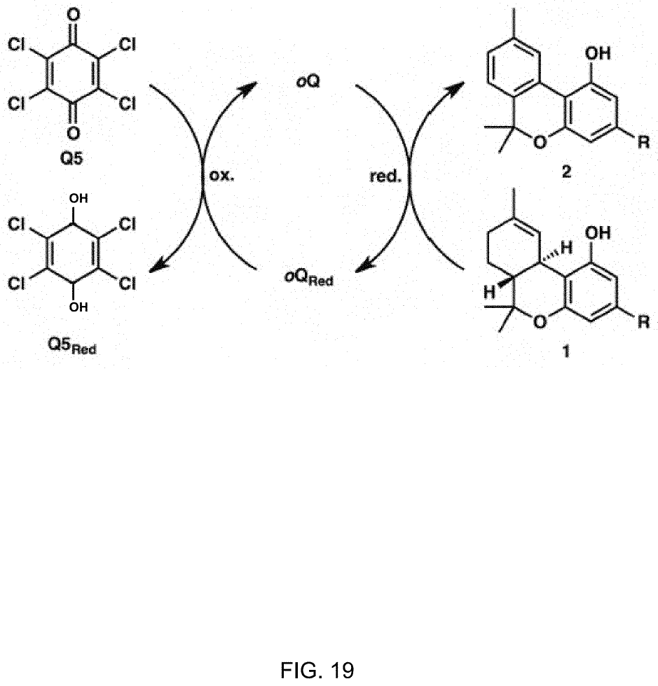

oQ and oIQ oxidation systems may be coupled to secondary oxidative processes to facilitate regeneration of the catalytic oxidant from its reduced catechol (oQRed) or 1,2-aminophenolic (oIQRed) form. This permits lowering of catalyst loading from stoichiometric (200 mol % and above) to catalytic concentrations (10-200 mol %, and non-zero amounts up to 10 mol %). As shown in FIG. 19, the dehydrogenation processes described can be catalyzed by 5 mol % of 3,5-di-tert-butyl-1,2-benzoquinone (Q3) when performed in the presence of the terminal oxidant p-chloranil (Q5) without negatively effecting reaction efficiency or outcomes.

A variety of redox couples may be employed in one pot for in situ regeneration of the oQ and oIQ oxidants. The catalysts may be re-oxidized using (for example) organic chemical oxidants such as p-chloranil (Q5), benzoquinone (Q1), N-oxide radicals such as (2,2,6,6-Tetramethylpiperidin-1-yl)oxyl (TEMPO), inorganic nitrite salts (e.g., NaNO₂), inorganic silver salts (e.g. Ag₂O), copper salts, chromium-based reagents, NaIO₄, and other hypervalent iodine species.

Additional inorganic and organic cocatalysts can facilitate aerobic oxidation. ZnI₂, I₂/DMSO, TBAI, Co(salophen) monohydrate, NaNO₂, and 1,4-quinones permit the re-oxidation of oQ and oIQ under an 02 atmosphere. In situ recycling of cannabinoid dehydrogenation catalysts may also be performed electrochemically, enzymatically, through photooxidation, or biologically.

Various aspects of different embodiments of the present disclosure are expressed in paragraphs X1, X2, X3, and X4 as follows:

X1: One embodiment of the present disclosure includes a method for aromatization of cannabinoids comprising applying an ortho-quinone to a cannabinoid in a solvent.

X2: Another embodiment of the present disclosure includes a method for reducing delta-9-tetrahydrocannabinol content of an extract of Cannabis sativa comprising applying to said extract an ortho-quinone and a solvent.

X3: A further embodiment of the present disclosure includes a method for chemically converting a first cannabinoid to a second cannabinoid comprising applying an ortho-quinone and a solvent to the first cannabinoid, wherein the first cannabinoid and the second cannabinoid are non-identical.

X4: Another embodiment of the present disclosure includes a method for reducing delta-9-tetrahydrocannabinol content of an extract of Cannabis sativa comprising mixing said extract with an ortho-quinone and a solvent.

Yet other embodiments include the features described in any of the previous paragraphs X1, X2, X3, or X4 combined with one or more of the following:

Wherein the solvent is at least one of acetonitrile, methylene chloride, toluene, methanol, ethanol, tetrahydrofuran, acetic acid, DMSO, chloroform, acetone, sulfuric acid, dichloromethane, and aliphatic hydrocarbons.

Wherein the solvent includes at least one of heptane, acetic acid, sulfuric acid, and dichloromethane.

Wherein the solvent is a mixture of acetic acid and sulfuric acid.

Wherein the solvent is a mixture of dichloromethane, acetic acid, and sulfuric acid.

Wherein the solvent is heptane.

Wherein the ortho-quinone is one of Q3, Q4, Q6, Q8, Q9, Q10, Q11, Q12, Q13, Q14, Q15, Q16, Q17, and Q18.

Wherein the ortho-quinone is one of Q3 and Q4.

Wherein the ortho-quinone is Q3.

Wherein the ortho-quinone is Q11.

Wherein the method further comprises applying Q5.

Wherein the ortho-quinone is an ortho-iminoquinone.

Wherein the cannabinoid is delta-9-tetrahydrocannabinol.

Wherein the applying has a duration in the range of 4 hours to 24 hours.

Wherein the mixing has a duration in the range of 4 hours to 24 hours.

Wherein the applying occurs at a temperature in the range of 20° C. to 40° C.

Wherein the applying occurs at a temperature in the range of 20° C. to 27° C.

Wherein, during the applying, a temperature is maintained in the range of 20° C. to 40° C.

Wherein, during the applying, a temperature is maintained in the range of 20° C. to 27° C.

Wherein the mixing occurs at a temperature in the range of 20° C. to 40° C.

Wherein the mixing occurs at a temperature in the range of 20° C. to 27° C.

Wherein, during the mixing, a temperature is maintained in the range of 20° C. to 40° C.

Wherein, during the mixing, a temperature is maintained in the range of 20° C. to 27° C.

Wherein the first cannabinoid is delta-9-tetrahydrocannabinol and wherein the second cannabinoid is cannabinol.

Wherein the first cannabinoid is cannabidiol and wherein the second cannabinoid is cannabinol.

Wherein the first cannabinoid is delta-8-tetrahydrocannabinol and wherein the second cannabinoid is cannabinol.

Wherein the first cannabinoid is delta-9-tetrahydrocannabivarin and wherein the second cannabinoid is cannabivarin and dihydrocannabivarin.

Wherein the first cannabinoid is delta-9-tetrahydrocannabinol and wherein the second cannabinoid is dihydrocannabinol.

Wherein the first cannabinoid is delta-9-tetrahydrocannabinol momomethyl ether and wherein the second cannabinoid is cannabinol monomethyl ether.

Wherein the first cannabinoid is 11-hydroxy-delta-9-tetrahydrocannabinol and wherein the second cannabinoid is 11-hydroxy-cannabinol.

Wherein the first cannabinoid is at least one of second cannabinoid is HU-345. tetrahydro derivative (26) under the previously described conditions. HU-3459 (29) may also be produced from HU-336 (27) or HU-331 (28

The foregoing detailed description is given primarily for clearness of understanding and no unnecessary limitations are to be understood therefrom for modifications can be made by those skilled in the art upon reading this disclosure and may be made without departing from the spirit of the invention. While examples, one or more representative embodiments, and specific forms of the disclosure, have been illustrated and described in detail in the drawings and foregoing description, the same is to be considered as illustrative and not restrictive or limiting. The description of particular features in one embodiment does not imply that those particular features are necessarily limited to that one embodiment. Some or all of the features of one embodiment can be used in combination with some or all of the features of other embodiments as would be understood by one of ordinary skill in the art, whether or not explicitly described as such. One or more exemplary embodiments have been shown and described, and all changes and modifications that come within the spirit of the disclosure are desired to be protected.

What is claimed is:

1) A method for aromatization of cannabinoids comprising applying an ortho-quinone to a cannabinoid in a solvent.

2) The method of claim 1, wherein the solvent is at least one of acetonitrile, methylene chloride, toluene, methanol, ethanol, tetrahydrofuran, acetic acid, DMSO, chloroform, acetone, sulfuric acid, dichloromethane, and aliphatic hydrocarbons.

3) The method of claim 2, wherein the solvent is at least one of heptane, acetic acid, sulfuric acid, and dichloromethane.

4) The method of claim 3, wherein the solvent is a mixture of acetic acid and sulfuric acid.

5) The method of claim 3, wherein the solvent is a mixture of dichloromethane, acetic acid, and sulfuric acid.

6) The method of claim 3, wherein the solvent is heptane.

7) The method of claim 1, wherein the ortho-quinone is one of Q3, Q4, Q6, Q8, Q9, Q10, Q11, Q12, Q13, Q14, Q15, Q16, Q17, and Q18.

8) The method of claim 7, wherein the ortho-quinone is one of Q3 and Q4.

9) The method of claim 8, wherein the ortho-quinone is Q3.

10) The method of claim 1, wherein the ortho-quinone is an ortho-iminoquinone.

11) The method of claim 1, wherein the cannabinoid is delta-9-tetrahydrocannabinol and the ortho-quinone is Q3.

12) The method of claim 1, wherein the applying has a duration in the range of 4 hours to 24 hours.

13) The method of claim 1, wherein the applying occurs at a temperature in the range of 20° C. to 40° C.

14) A method for reducing delta-9-tetrahydrocannabinol content of an extract of Cannabis sativa comprising applying to said extract an ortho-quinone and a solvent.

15) The method of claim 15, wherein the solvent is at least one of acetonitrile, methylene chloride, toluene, methanol, ethanol, tetrahydrofuran, acetic acid, DMSO, chloroform, acetone, sulfuric acid, dichloromethane, and aliphatic hydrocarbons.

16) The method of claim 15, wherein the ortho-quinone is one of Q3, Q4, Q6, Q8, Q9, Q10, Q11, Q12, Q13, Q14, Q15, Q16, Q17, and Q18.

17) A method for chemically converting a first cannabinoid to a second cannabinoid comprising applying an ortho-quinone and a solvent to the first cannabinoid, wherein the first cannabinoid and the second cannabinoid are non-identical.

18) The method of claim 18, wherein the solvent is at least one of acetonitrile, methylene chloride, toluene, methanol, ethanol, tetrahydrofuran, acetic acid, DMSO, chloroform, acetone, sulfuric acid, dichloromethane, and aliphatic hydrocarbons.

19) The method of claim 18, wherein the ortho-quinone is one of Q3, Q4, Q6, Q8, Q9, Q10, Q11, Q12, Q13, Q14, Q15, Q16, Q17, and Q18.

20) The method of claim 18, wherein the first cannabinoid is delta-9-tetrahydrocannabinol and wherein the second cannabinoid is cannabinol.
